(19) United States
(12) Patent Application Publication
(10) Pub. No.: US 2013/0030344 A1
(43) Pub. Date: Jan. 31, 2013

(54) METHOD AS WELL AS APPARATUSES FOR REMOVING GAS ACCUMULATIONS FROM A COMPONENT OF AN EXTRACORPOREAL BLOOD CIRCUIT

(71) Applicants: Soeren Gronau, Nauheim (DE); Dejan Nikolic, Frankfurt (DE); Joachim Noack, Bad Neustadt (DE)

(72) Inventors: Soeren Gronau, Nauheim (DE); Dejan Nikolic, Frankfurt (DE); Joachim Noack, Bad Neustadt (DE)

(73) Assignee: FRESENIUS MEDICAL CARE DEUTSCHLAND GMBH, Bad Homburg (DE)

(21) Appl. No.: 13/560,184

(22) Filed: Jul. 27, 2012

Related U.S. Application Data
(60) Provisional application No. 61/512,933, filed on Jul. 29, 2011.

(30) Foreign Application Priority Data
Jul. 29, 2011 (DE) 10 2011 110 472.4

(51) Int. Cl. A61M 1/36; B01D 61/32

(52) U.S. Cl. 604/5.01; 210/646; 210/143; 137/1; 137/561 R

(57) ABSTRACT

The present invention relates to a method for removing gas accumulations from a component of an extracorporeal blood circuit, a control device for executing a method according to the present invention, a medical treatment apparatus which comprises at least one control device and/or stands in signal transmission or is connected for signal transmission with it, a digital storage medium, a computer program product as well as a computer program.

### CROSS REFERENCE TO RELATED APPLICATIONS

This application claims the benefit of U.S. Provisional Patent Application No. 61/512,933 filed on Jul. 29, 2011 and German Patent Application No. 10 2011 110 472.4, filed Jul. 29, 2011, both of which are hereby incorporated by reference in their entireties.

### FIELD OF INVENTION

The present invention relates to a method for removing gas accumulations from a component of an extracorporeal blood circuit through which fluid flows during an extracorporeal blood treatment or which comes in contact with the flow in a first flow direction. In addition, it relates to a control device, a medical treatment apparatus, a digital storage medium, a computer program product, and a computer program for performing such methods.

### BACKGROUND OF INVENTION

Extracorporeal blood tube systems as are used, e.g., during dialysis or during the use of heart-lung machines often have devices for holding back blood clots. These so-called clot catchers usually are on the output side of the blood tube system and are regularly embodied as finely woven tissue or fabric or grid. At its meshes, gas bubbles may get caught, which is undesirable. Methods and constructive measures are known for preventing or reducing gas bubbles getting caught at the clot catcher and/or for removing gas accumulations or gas adhesions from the clot catcher.

One object of the present invention is to propose a further method for removing gas bubbles or gas accumulations from a clot catcher or another component or element (hereafter component) of an extracorporeal blood circuit. In addition, suitable devices or apparatuses, a suitable digital storage medium, a suitable computer program product and a suitable computer program are to be specified.

Therefore, according to the present invention a method is proposed for removing gas accumulations from (or out of) an extracorporeal blood circuit's clot catcher or another component through which fluid flows during an extracorporeal blood treatment or which comes in contact with the flow in a first flow direction. The method according to the present invention encompasses generating a flow of a fluid through the component, e. g. the clot catcher,—or into it—in a second flow direction.

The control device according to the present invention is suitable and provided and/or construed and/or configured for executing the method according to the present invention.

The medical or medical-technical treatment apparatus according to the present invention (hereafter referred to as treatment apparatus) comprises at least one control device and/or is connected in signal transmission herewith or is in a signal transmission relation herewith.

A digital, particularly a non-volatile storage medium according to the present invention, particularly in the form of a machine-readable data storage device, particularly in the form of a disk, CD EPROM or DVD, with electronically or optically readable control signals may interact with a programmable computer system such that the mechanical steps of the method according to the present invention are prompted.

In doing so, all or some of the mechanically executed steps of the method according to the present invention may be prompted.

A computer program product according to the present invention comprises a program code—e.g., volatile, transitory, or non-transitory and saved on a machine-readable storage device—for prompting the mechanical steps of the method according to the present invention when the computer program product runs on a computer. According to the present invention a computer program product can be understood as, for example, a computer program which is stored on a storage device, an embedded system as a comprehensive system with a computer program (e.g., an electronic device with a computer program), a network of computer-implemented computer programs (e.g., a client-server system, a cloud computing system, etc.), or a computer on which a computer product is loaded, executed, saved or developed.

The term “machine-readable storage device,” as used herein, denotes in certain embodiments of the present invention a storage device which contains data or information which is interpretable by software and/or hardware. The storage device may be a data storage device such as a disk, a CD, DVD, a USB stick, a flashcard, an SD card and the like.

A computer program according to the present invention comprises a program code for prompting the mechanical steps of the method according to the present invention when the computer program runs on a computer. A computer program according to the present invention can be understood as, for example, a physical software product, which is ready for distribution and contains a computer program.

It also applies for the computer program product according to the present invention and the computer program according to the present invention that all or some of the mechanically executed steps of the method according to the present invention are prompted.

In all of the following embodiments, the use of the expression “may be” or “may have” and so on, is to be understood synonymously with “preferably is” or “preferably has,” respectively, and so on, and is intended to illustrate an embodiment according to the present invention.

Advantageous developments of the present invention are each subject of dependent claims and embodiments.

Embodiments according to the present invention may comprise one or more of the features named hereafter.

In some embodiments according to the present invention, a gas accumulation is air or a different gaseous mixture.

In some embodiments according to the present invention, the fluid is a liquid, preferably a dialyzing liquid or substituate. In some embodiments according to the present invention, the fluid is a hydraulic liquid, e.g., from the compliance of the hydraulics (dialysate side) of the treatment apparatus.

In certain embodiments according to the present invention, generating a flow of a fluid takes place through the apertures of the clot catcher.

The second flow direction is in some embodiments according to the present invention not the first flow direction. In certain embodiments according to the present invention, the second flow direction is opposite to the first flow direction. In some embodiments according to the present invention, a fluid which moves in the second flow direction flows into or towards the venous blood chamber.

In some embodiments according to the present invention, the method encompasses building up pressure by means of the fluid at a time which is before the time of generating the flow or of the beginning of the flow in the second flow direction. In these embodiments, at first a pressure is built up the balance of which or the at least partial pressure release of which is allowed only after a predefined or desired pressure level is reached, namely by means of or by generating a fluid flow in the second flow direction. Building up pressure by means of the fluid therefore takes place chronologically before generating the flow in the second flow direction.

The pressure built up this way is in some embodiments according to the present invention built up in front of or before a valve.

The pressure built up by means of the fluid is in some embodiments according to the present invention a positive pressure.

In some embodiments according to the present invention, this pressure is an overpressure. In some embodiments according to the present invention, this pressure is higher than a usual or average operating pressure which is present while flowing through the clot catcher in the first flow direction or during the blood treatment.

In other embodiments according to the present invention, the pressure is a negative pressure, e.g., across the membrane of the blood filter.

The negative pressure may be a pressure drop originating from the blood compartment of the blood filter in the direction of the dialysate compartment of the blood filter.

The negative pressure may arise or may have arisen through a suction of the hydraulics of the treatment apparatus.

In certain embodiments according to the present invention, the pressure is above a pressure generated by means of the conveying device during the blood treatment or average pressure.

Building up pressure by means of the fluid corresponds in some embodiments according to the present invention to a sudden pressure increase in or at the clot catcher. A sudden pressure increase is in certain embodiments exclusively due to the use of the conveying device, in other embodiments, however, it is not.

In some embodiments according to the present invention, the method encompasses generating the flow in the second direction and/or building up the pressure by using a blood pump. In these or in other embodiments according to the present invention, the method encompasses generating the flow in the second direction and/or building up the pressure by using a hydraulic device of a medical treatment apparatus.

In some embodiments according to the present invention, the method encompasses closing at least one valve to support the build-up of the pressure.

In certain embodiments according to the present invention, closing is an active process, and/or closing takes place for the purpose of supporting the build-up of the pressure.

The at least one valve to be closed is in some embodiments according to the present invention is a venous patient tube clamp.

In certain embodiments according to the present invention, the method encompasses bypassing in the sense of interconnecting by creating a fluid communication between an arterial section of the extracorporeal blood circuit with a venous section of the extracorporeal blood circuit. In other embodiments according to the present invention, the method does not encompass such bypassing or connecting.

In some embodiments according to the present invention, the method encompasses monitoring the level or head of the built-up pressure and/or of the pressure gradient by means of a suitable device.

Monitoring the level or head of the built-up pressure and/or of the pressure gradient is in certain embodiments according to the present invention accompanied by or encompasses a delimitation of the level or head.

In some embodiments according to the present invention, monitoring the level or head of the built-up pressure and/or of the pressure gradient takes place by opening the valve or the clamp when or after a predefined maximum level or head of the pressure is reached.

The device for monitoring the level or head of the built-up pressure and/or of the pressure gradient comprises or is in certain embodiments according to the present invention a pressure sensor.

In some embodiments according to the present invention, the method encompasses opening the valve of the addition point for substituate liquid for predilution and/or the valve of the addition point for substituate liquid for postdilution. Opening this valve may advantageously contribute to advantageously circumventing the flow resistance of the blood filter or dialyzer.

In certain embodiments according to the present invention, the method encompasses opening the single-needle valve, in some embodiments according to the present invention it encompasses opening the vent valve of the extracorporeal blood circuit. Opening this valve may advantageously contribute to preventing a large or even excessive build-up of negative pressure on the suction side of the conveying device. In other embodiments according to the present invention, the single-needle valve and/or the vent valve of the extracorporeal blood circuit remain closed. This may advantageously contribute to reaching high peak pressures and/or a high pressure gradient across the clot catcher.

In certain embodiments according to the present invention, the method encompasses executing all steps mentioned before twice or several times.

The method encompasses in some embodiments according to the present invention cancelling the method after a predefined number of performed cycles and/or after a predefined volume of fluid is conveyed in the second flow direction by means of the conveying device.

In certain embodiments according to the present invention, the method takes place before the beginning of a blood treatment, e.g., in connection with rinsing or priming the extracorporeal blood circuit.

“Before the beginning of a blood treatment” means in some embodiments according to the present invention that the method according to the present invention is finished before the beginning of the patient's treatment.

In certain embodiments according to the present invention, the control device comprises setting devices, measuring devices, control or regulating devices, evaluating devices, comparing devices, and/or memory devices for comparative data.

The treatment apparatus according to the present invention is in some embodiments embodied as blood treatment apparatus, in particular as an apparatus for aphaeresis or dialysis, again in particular for hemodialysis, hemofiltration, hemodiafiltration, or for acute dialysis.

In some embodiments according to the present invention of the treatment apparatus, the conveying device is embodied as a blood pump. In certain embodiments according to the present invention, it is embodied as a roller pump.

The present invention relates to a tube configuration and a method which advantageously facilitates deairing clot catchers: The method is based on the flow direction being temporarily reversed, e.g., while filling/rinsing the disposable in the clot catcher.

In certain embodiments according to the present invention removing gas bubbles or gas accumulations by the method according to the present invention or by flushing free is possible in any section or position of the extracorporeal blood circuit and from any component of the extracorporeal blood circuit. Those sections or positions or components include the blood filter or the dialyzer. The points or positions or components especially include those, through which—or into which—a pressure wave created according to the present invention flows. The present invention is therefore not limited to removing gas bubbles or gas accumulations from the clot catcher, even though parts of the description are directed to a clot catcher which, however, should not be understood as limiting. Gas bubbles or gas assemblies can also be removed by the method according to the present invention from other components or parts of the extracorporeal blood circuit or of the blood tube set, as e. g. the blood filter. Hence, the present invention encompasses this as well.

Some or all embodiments according to the present invention may comprise one or more of the advantages named above or hereafter.

Thus, in contrast to providing state of the art coatings which are supposed to improve the deaeration properties of the clot catcher by modifying the wettability of the material, the method according to the present invention advantageously does not enhance the ability of the air bubbles to pass through the clot catcher; the latter would be undesired during forward operation.

The method may be performed during the preparation of the disposable, e.g., in the form of an extracorporeal blood circuit, or of the treatment apparatus without the user having to intervene.

Eliminating remaining air advantageously prevents a possible undesired air infusion during the treatment (induced, e.g., by applying blood flow speeds during the treatment which are higher than the flow speeds during the preparation of the apparatuses).

Eliminating remaining air in the extracorporeal system may also advantageously improve the hemocompatibility of the whole system.

The control of the ventilation by means of pressures occurring when the clamp is closed advantageously prevents the tube system from bursting. This is relevant because tube roller pumps possibly have significantly higher occlusion pressures during reverse flow than during normal operation.

The function of the arterial pressure measurement in the over-pressure range may also be tested as part of the reverse flow: For this, the single-needle valve could be closed, e.g., before opening the venous clamp. Then, a positive residual pressure develops in the whole system; this pressure may be determined and compared by means of arterial and venous pressure measurement.

The consequences of a hampered transfer of residual air through the meshes of a clot catcher according to the present invention, e.g., due to existing lift, may also be advantageously counteracted according to the present invention.

### BRIEF DESCRIPTION OF THE DRAWINGS

The present invention is hereafter exemplarily explained by means of the appended drawings in which identical reference numerals refer to identical or similar components. In the in part highly simplified figures it applies that:

FIG. 1 shows an example of a blood clot catcher in a sectional view from which gas adhesions may be removed by means of the method according to the present invention;

FIG. 2 shows a schematically simplified extracorporeal blood circuit for removing gas adhesions from a blood clot catcher.

### DETAILED DESCRIPTION

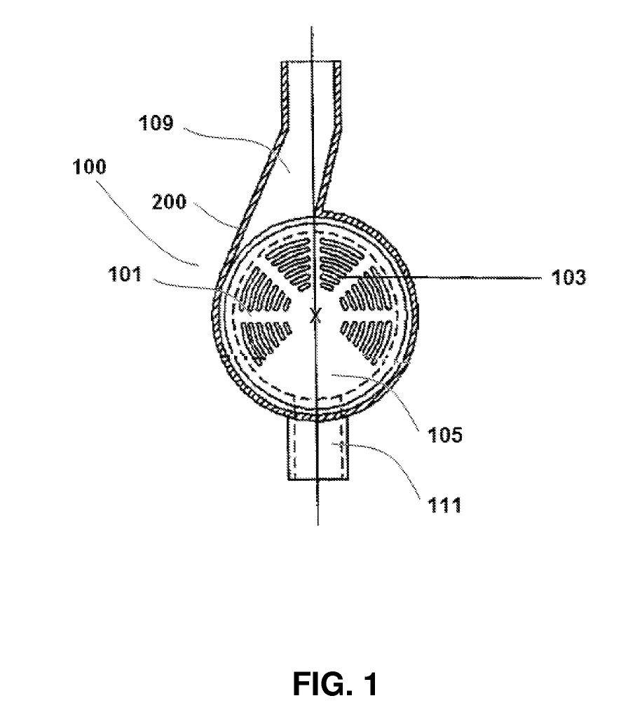

FIG. 1 shows a section through a blood clot catcher (in short also: clot catcher) 100 arranged in a housing 200. The clot catcher 100 comprises a screen surface 101 which comprises apertures 103 and a section 105 without such apertures. The inflow area of the clot catcher 100 is marked with the reference numeral 109, the outflow area with 111. Air bubbles may adhere to the apertures 103. These may be removed from the apertures by means of the method according to the present invention.

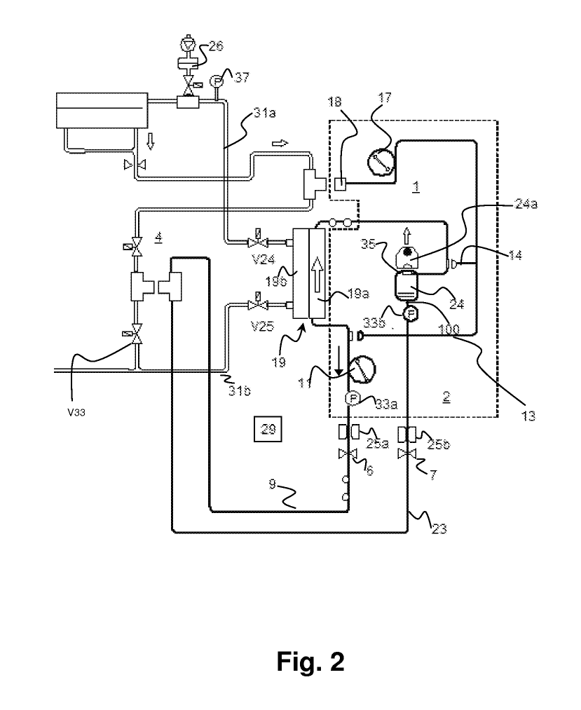

FIG. 2 shows a schematically simplified extracorporeal blood circuit 1 which at least in sections hereof is part of a blood cassette 2. The blood cassette 2 is inserted into a medical blood treatment apparatus (in short also: apparatus) 4 for the treatment of the patient.

The extracorporeal blood circuit 1 comprises an arterial patient tube clamp 6 of an arterial section 9, further a venous patient tube clamp 7 of a venous section 23.

A blood pump 11 is provided in the arterial section 9, a substituate pump 17 is connected with a substituate line. The substituate line may be connected with a source of substituate by means of an automatic substituate port 18. Substituate may be introduced into line sections of the blood circuit 1 via predilution or via postdilution and the appropriate addition points 13 or 14 by means of the substituate pump 17.

A blood filter 19 is connected with the blood circuit 1. The blood filter 19 comprises a blood chamber 19a which is connected with the arterial section 9 and with the venous section 23. A dialysate chamber 19b is connected with a dialysate inlet line 31a which leads to the dialysate chamber 19b and a dialysate outlet line 31b which leads away from the dialysate chamber 19b. The venous section 23 is in fluid communication with a venous blood chamber 24 of the extracorporeal blood circuit 1.

The venous blood chamber 24 is in fluid connection with the clot catcher 100 at its outflow side and/or comprises such a clot catcher 100.

The venous blood chamber 24 is in fluid connection with a single-needle valve 35 (SN valve) at its inflow side or comprises such a single-needle valve 35. The venous blood chamber 24 is furthermore in fluid connection with a vent valve 24a at its inflow side or comprises such a vent valve 24a.

Air detectors 25a and 25b are provided in the arterial section 9 or in the venous section 23.

A control device 29 according to the present invention is schematically illustrated. It is in contactless signal connection (touchless) and/or in contact connection with the components required for executing the method according to the present invention such as sensors, clamps, valves, blood pump and so on.

The dialysate inlet line 31a comprises a valve V24 by means of which the flow within the dialysate inlet line 31a may be stopped. The dialysate outlet line 31b comprises a valve V25 by means of which the flow within the dialysate outlet line 31b may be stopped.

The dialysate inlet line 31a is further connected with a source of compressed air 26 by means of a further, machine-internal valve.

The pressure sensors 33a and 33b measure the pressure in the arterial section 9 or in the venous section 23. A pressure sensor 37 measures the pressure in the dialysate inlet line 31a.

The dialysate circuit comprises a rinsing valve V33.

For executing the method according to the present invention, in certain embodiments, the arterial section 9 and the venous section 23 are bypassed in arrangements such as are shown in FIG. 2. The rinsing valve V33 may be closed. Subsequently, the rotating direction of the blood pump 11 is reversed. The blood pump 11 conveys liquid against the venous patient tube clamp 7, which was closed in the meantime, in the direction indicated in FIG. 2 with the arrow. The pressure which is built-up hereby is delimited through the pressure sensor present in the apparatus 4: When a predetermined maximum pressure is reached, the venous patient tube clamp 7 is opened. The pressure caught in the compliance between blood pump 11 and venous patient tube clamp 7 is abruptly released into the venous blood chamber 24. It may there lead to partially high and very high peak flows which are significantly larger than the flows which occur when the blood pump 11 conveys in the first conveying direction during normal operation of the apparatus 4. Due to the peak flows, air bubbles that are present on the output side (in FIG. 2 at the lower end of the clot catcher) of the clot catcher 100 are carried away completely or at least partially across the narrow passages of the clot catcher 100.

In the embodiment of the method according to the present invention which is proposed here, a use of the substituate pump 17 is not provided and/or not necessary. The valves of the addition points 13 and 14 for substituate liquid in predilution or postdilution (also denoted as pre-valve and post-valve) are opened in order to evade the flow resistance of the blood filter 19, also denoted as dialyzer. The single-needle valve 35 and the vent valve 24a are open in order to prevent a strong build-up of negative pressure on the suction side of the blood pump. In order to increase the peak flows, the single-needle valve 35 and/or the vent valve 24a may also be closed in order to further increase the pressure gradient across the clot catcher 100.

The deaeration procedure described above may—if necessary—be repeated once or several times.

The method according to the present invention is especially effective with a positioning of the clot catcher such that air bubbles accumulate at a point at which they may be carried away by the flow in an especially unhindered way when the flow is reversed.

In a further embodiment according to the present invention, rinsing the clot catcher 100 takes place by means of a liquid which is taken from the hydraulics or hydraulic device of the treatment apparatus 4 for this purpose. In doing so, the valves V24 and/or V25 may be open. At the same time, the single-needle valve 35 and/or the vent valve 24 may be closed. Also in this embodiment, the fluid is conveyed by means of the blood pump 11 which conveys backwards or in the second flow direction. If necessary, it also builds up the overpressure described above.

The further embodiment according to the present invention described hereafter differs from this. In this embodiment, the arterial section 9 and the venous section 23 are advantageously not being bypassed or connected—different from what is shown in FIG. 2—(even though this is quite possible and also encompassed by the present invention also in this embodiment, e.g., with the blood pump 11 running backwards). In this embodiment, building up the required pressure of the fluid in the clot catcher 100 takes place by means of a negative pressure at the membrane of the blood filter 19 which is achieved by the use of the hydraulics of the treatment apparatus 4.

What is claimed is:

1. A method for removing gas accumulations from a component of an extracorporeal blood circuit through which fluid flows during an extracorporeal blood treatment or which comes in contact with the flow in a first flow direction, said method comprising the step of:
generating a flow of a fluid through or into the component in a second flow direction.

2. The method according to claim 1, further comprising the step of:
building up pressure by means of the fluid before generating the flow in the second flow direction.

3. The method according to claim 2, further comprising the step of:
closing at least one valve in order to support the pressure build-up.

4. The method according to claim 1, further comprising the step of:
bypassing an arterial section of the extracorporeal blood circuit with a venous section of the extracorporeal blood circuit.

5. The method according claim 2, further comprising the step of:
generating the flow in the second direction and/or building up the pressure or negative pressure using a blood pump and/or using a hydraulic device of a medical treatment apparatus.

6. The method according to claim 2, further comprising the step of:
using a pressure monitoring device to monitor the level or head of the built-up pressure and/or of a pressure gradient.

7. The method according to claim 1, further comprising the step of:
opening a valve of an addition point for substituate liquid for predilution and/or a valve of an addition point for substituate liquid for postdilution or one or more further valves for opening a communication connection with a blood filter.

8. The method according to claim 1, further comprising the step of:
opening a single-needle valve.

9. The method according to claim 1, further comprising the step of:
opening a vent valve.

10. The method according to claim 5, further comprising the step of:
conveying fluid across a membrane of a blood filter by utilizing the hydraulic device of the treatment apparatus as a conveying device.

11. The method according to claim 10, wherein a negative pressure is produced across the membrane of the blood filter by means of the hydraulic device of the treatment apparatus.

12. The method according to claim 11, further comprising the step of:
executing all steps of claim 11 twice.

13. The method according to claim 12, further comprising the step of:
cancelling the method after a predefined number of performed cycles and/or after a predefined volume of fluid was conveyed in the second flow direction by means of the conveying device.

14. The method according to claim 1 further comprising the step of:
executing the method before the beginning of a blood treatment session.

15. A system for executing a method according to claim 1, said system comprising a control device configured to execute the method according to claim 1.

16. The system according to claim 15, said system further comprising a medical treatment apparatus which comprises the control device and/or stands in signal transmission or is connected for signal transmission with said control device.

17. The system according to claim 16, wherein the system is configured as an apparatus for hemodialysis, hemofiltration, hemodiafiltration, or for acute dialysis.

18. A non-transitory computer-readable medium with an executable program stored thereon, wherein the program is configured to instruct a programmable computer system to execute the method according to claim 1.
